(19) United States
(12) Patent Application Publication
(10) Pub. No.: US 2005/0004111 A1
(43) Pub. Date: Jan. 6, 2005

(54) Selective MMP-13 inhibitors

(75) Inventors: Otmar Klingler, Rodgau (DE); Reinhard Kirsch, Braunschweig (DE); Joerg Habermann, Roma (IT); Klaus-Ulrich Weithmann, Hofheim (DE); Christian Engel, Frankfurt (DE); Bernard Pirard, Darmstadt (DE)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: AVENTIS PHARMA DEUTSCHLAND GMBH, Frankfurt am Main (DE)

(21) Appl. No.: 10/751,545

(22) Filed: Jan. 5, 2004

Related U.S. Application Data
(60) Provisional application No. 60/472,765, filed on May 22, 2003.

(30) Foreign Application Priority Data
Jan. 3, 2003 (DE) 10300017.8

(51) Int. Cl. A61K 31/541; A61K 31/5377; A61K 31/506; C07D 417/02; C07D 413/02; C 07D 4 3/02

(52) U.S. Cl. 514/227.5; 514/235.5; 514/252.14; 514/256; 544/60; 544/122; 544/295; 544/333

(57) ABSTRACT

The invention relates to a pyrimidine-4,6-dicarboxylic acid diamide compound, pharmaceutical preparation comprising it, process for preparing it and method for its pharmaceutical use. Particularly, the pyrimidine-4,6-dicarboxylic acid diamide compound is useful for selectively inhibiting collagenase matrix metalloproteinase (MMP) 13, or for treating a degenerative joint disease.

### FIELD OF THE INVENTION

The invention relates to a pyrimidine-4,6-dicarboxylic acid diamide compound, pharmaceutical preparation comprising it, process for preparing it and method for its pharmaceutical use. Particularly, the pyrimidine-4,6-dicarboxylic acid diamide compound is useful for selectively inhibiting collagenase matrix metalloproteinase (MMP) 13, or for treating a degenerative joint disease.

### BACKGROUND OF THE INVENTION

In diseases such as osteoarthritis and rheumatism, destruction of the joint takes place, with this destruction being caused, in particular, by the proteolytic breakdown of collagen due to collagenases. Collagenases belong to the metalloproteinase (MP) or MMP superfamily. Under physiological conditions, MMPs cleave collagen, laminin, proteoglycans, elastin or gelatin and therefore play an important role in bone and connective tissue. A large number of different inhibitors of the MMPs and/or collagenases have been is closed (EP 0606046; WO94/28889). Known MMP inhibitors frequently suffer from the disadvantage of lacking the specificity involved in inhibiting only one class of MMPs. As a result, most MMP inhibitors inhibit several MMPs simultaneously because the structure of the catalytic domain in the MMPs is similar. As a consequence, the inhibitors have the undesirable property of acting on many enzymes including those that have a vital function (Massova I., et al., The FASEB Journal (1998) 12, 1075-1095).

It is known that pyrimidine-4,6-dicarboxylic acid diamides and 2,4-substituted pyridine N-oxides inhibit the enzymes proline hydroxylase and lysine hydroxylase and thereby bring about an inhibition of collagen biosynthesis by exerting an influence on the collagen-specific hydroxylation reaction (EP 0418797; EP 0463592). This inhibition of collagen biosynthesis results in the formation of a nonfunctional, under-hydroxylated collagen molecule that the cells can only release into the extracellular space in small quantity. In addition, the under-hydroxylated collagen cannot be incorporated into the collagen matrix and is very readily degraded proteolytically. As a consequence of these effects, the overall quantity of collagen that is deposited extracellularly decreases. It is known from patent applications WO 02/064571 and WO 02/064080 that certain pyridine-2,4-dicarboxylic acid diamides and pyrimidine-4,6-dicarboxylic acid diamides can be allosteric inhibitors of MMP 13.

WO 02/064571 describes pyrimidine-4,6-dicarboxylic acid and pyrimidine-4,6-dicarboxylic acid derivatives and their selective inhibition of MMP 13. EP 0418797 also describes pyrimidine-4,6-dicarboxylic acid derivatives and their inhibitory effect on proline hydroxylase. It was possible to confirm this inhibition by pyrimidine-4,6-dicarboxylic acid in in-house experiments. The compounds described in WO 02/064571 and EP 0418797 therefore suffer from the disadvantage that, as a result of proline hydroxylase being inhibited, collagen biosynthesis is also inhibited and a nonfunctional, under-hydroxylated collagen molecule is formed, with the cells only being able to release this molecule into the extracellular space in small quantities.

In view of the current situation, it is clear that there is a compound that selectively inhibits MMP 13 but does not inhibit proline hydroxylase, and is therefore better suited for more efficiently and more specifically treating a degenerative joint disease.

### SUMMARY OF THE INVENTION

The invention is directed to a powerful inhibitor, of MMP 13 that has essentially no activity on the MMPs 3 and 8 and does not inhibit proline hydroxylase.

The invention therefore relates to the compound of formula I 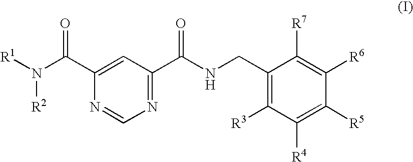
wherein
R¹ is
   hydrogen atom or —(C₁-C₆)-alkyl,
R² is
   —(C₁-C₆)-alkyl that is substituted, once, twice or three times, by
      —C(O)—O—R⁸,
      —(C₁-C₆)-alkyl-O—R⁸,
      —(C₆-C₁₄)-aryl that is substituted, once, twice or three times, independently of each other, by R¹¹ or
      Het that is a saturated or unsaturated monocyclic or bicyclic, 3- to 10-membered heterocyclic ring system which contains 1, 2 or 3 identical or different ring heteroatoms selected from the group consisting of nitrogen, oxygen and sulfur and is unsubstituted or substituted, once or more than once, by R¹³,
R³, R⁴, R⁵, R⁶ and R⁷ are identical or different and are, independently of each other,
   hydrogen
   halogen,
   —(C₁-C₆)-alkyl, in which alkyl is unsubstituted or substituted, once, twice or three times, by halogen,
   —O—(C₁-C₆)-alkyl, in which alkyl is-unsubstituted or substituted, once, twice or three times, by halogen, or
   —S—(C₁-C₆)-alkyl,
R⁸ is
   hydrogen atom, or
   —(C₁-C₆)-alkyl,
R¹¹ is
   —(C₂-C₆)-alkyl-C(O)—O—R⁸,
   —O—(C₁-C₆)-alkyl-C(O)—O—R⁸,
   —NR¹⁴R¹⁵,
   —(CH₂)ₖ—NR⁹R¹⁰,
   —O—(C₂-C₆)-alkyl-NR⁹R¹⁰, or
   —NR⁸—C(O)—(C₁-C₆)-alkyl, in which alkyl is unsubstituted or substituted, once, twice or three times, by R¹²,
R⁹ and R¹⁰ are identical or different and are, independently of each other,
   hydrogen atom, or
   —(C₁-C₆)-alkyl, or
   taken together with the nitrogen atom to which they are attached form a 5-, 6- or 7-membered saturated azaheterocyclyl ring wherein one or two further carbon atoms thereof are optionally replaced by a heteroatom that is an oxygen, sulfur or nitrogen atom, and wherein the nitrogen atom is optionally unsubstituted or substituted by (C₁-C₆)-alkyl,
k is
   2, 3, 4 or 5,
R¹² is
   halogen,
   cyano,
   nitro,
   hydroxyl,
   amino,
   —C(O)—O—(C₁-C₆)-alkyl, or
   —C(O)—OH,
R¹³ is
   halogen,
   cyano,
   nitro,
   hydroxyl,
   amino,
   —C(O)—O—(C₁-C₆)-alkyl,
   —C(O)—OH,
   —(C₁-C₆)-alkyl that is unsubstituted or substituted, once, twice or three times, by halogen,
   —O—(C₁-C₆)-alkyl, where alkyl is unsubstituted or substituted, once, twice or three times, by halogen,
   pyridyl, or
   phenyl that is unsubstituted or substituted, once or more than once and independently of each other, by a radical from the series halogen, (C₁-C₆)-alkoxy and (C₁-C₆)-alkyl, and

R¹⁴ and R¹⁵ together with the nitrogen atom to which they are attached form a 5-, 6- or 7-membered saturated azaheterocyclyl ring wherein one or two further carbon atoms thereof are optionally replaced by a heteroatom that is oxygen, sulfur or nitrogen, and wherein each nitrogen atom thereof is optionally independently unsubstituted or substituted by (C₁-C₆)-alkyl, or
stereoisomer thereof, a mixture of stereoisomers thereof in any ratio, or physiologically tolerable salt thereof.

The invention also is directed to the use of the compound of formula I for the prophylaxis or therapy of a patient having or subject to a disease that involves a detrimental increase in the activity of matrix metalloproteinase 13.

### DETAILED DESCRIPTION OF THE INVENTION

Definitions

As used above, and throughout the description of the invention, the following terms, unless otherwise indicated, shall be understood to have the following meanings:-

The term “halogen” means fluorine, chlorine, bromine or iodine.

The term “—(C₁-C₆)-alkyl” means, in the widest possible sense, hydrocarbon radicals containing 1, 2, 3, 4, 5 or 6 carbon atoms and whose carbon chain is straight or branched or which consist of cyclic hydrocarbon groups or of combinations of linear and cyclic groups. For example, linear and branched hydrocarbon radicals can be methyl, ethyl, propyl, i-propyl, butyl, tertiary butyl, pentyl or hexyl, while cyclic groups can be cyclopropyl, cyclobutyl, cyclopentyl or cyclohexyl, and a combination of linear and cyclic radicals can be cyclopropylmethyl, cyclobutylmethyl or cyclopentylmethyl. The phrase “R⁴ and R⁵ or R⁵ and R⁶ form, together with the carbon atoms to which they are in each case bonded, a 5- or 6-membered ring which is aromatic or saturated and contains zero, one or two heteroatoms from the series oxygen, nitrogen or sulfur” means radicals which can be derived, for example, from dioxolane, pyrrole, pyrrolidine, pyridine, piperidine, dioxane, tetrahydropyridine, pyrazole, imidazole, pyrazoline, imidazoline, pyrazolidine, imidazolidine, pyridazine, pyrimidine, pyrazine, piperazine, pyran, furan, dihydrofuran, tetrahydrofuran, oxazole, isoxazole, 2-isoxazoline, isoxazolidine, morpholine, oxothiolane, thiopyran, thiazole, isothiazole, 2-isothiazoline, isothiazolidine or thiomorpholine.

The term “—(C₆-C₁₄)-aryl” means aromatic carbon radicals having from 6 to 14 carbon atoms in the ring. Examples of —(C₆-C₁₄)-aryl radicals are phenyl, naphthyl, for Example 1-naphthyl and 2-naphthyl, biphenylyl, for Example 2-biphenylyl, 3-biphenylyl and 4-biphenylyl, anthryl or fluorenyl. Biphenylyl radicals, naphthyl radicals and, in particular, phenyl radicals are preferred aryl radicals.

The term “Het” means a saturated or unsaturated monocyclic or bicyclic, 3- to 10-membered heterocyclic ring system which contains 1, 2 or 3 identical or different ring heteroatoms from the series nitrogen, oxygen and sulfur. In the underlying monocyclic or bicyclic heterocyclic ring system, Het contains 3, 4, 5, 6, 7, 8, 9 or 10 ring atoms. The monocyclic ring system can be a 3-, 4-, 5-, 6- or 7-membered ring. In the bicyclic Het, two rings can be linked to each other, with it being possible for one of the rings to be a 5-membered or 6-membered heterocyclic ring and the other to be a 5- or 6-membered heterocyclic or carbocyclic ring. A bicyclic Het group can, for example, be composed of 8, 9 or 10 ring atoms.

Het comprises saturated heterocyclic ring systems which do not possess any double bond in the rings and also unsaturated heterocyclic ring systems, including monounsaturated and polyunsaturated heterocyclic ring systems, which possess one or more double bonds and form a stable ring system. Unsaturated rings can be partially unsaturated or form an aromatic system. The Het group contains identical or different heteroatoms from the series nitrogen, oxygen and sulfur. Examples of heterocycles from which the Het group can be derived are the radicals acridinyl, benzimidazolyl, benzofuranyl, benzothiofuranyl, benzothiophenyl, benzoxazolyl, benzothiazolyl, benzotriazolyl, benzotetrazolyl, benzisoxazolyl, benzisothiazolyl, benzimidazalinyl, carbazolyl, 4aH-carbazolyl, carbolinyl, chromanyl, chromenyl, cinnolinyl, decahydroquinolinyl, 2H, 6H-1,5,2-dithia-zinyl, dihydrofuran[2,3-b]tetrahydrofuran, fuaranyl, furazanyl, imidazolidinyl, imidazolinyl, imidazolyl, 1H-indazolyl, indolinyl, indolizinyl, indolyl, 3H-indolyl, isobenzofuranyl, isochromanyl, isoindazolyl, isoindolinyl, isoindolyl, isoquinolinyl (benzimidazolyl), isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purynyl, pyranyl, pyrazinyl, pyroazolidinyl, pyrazolinyl, pyrazolyl, pyridazinyl, pyridooxazoles, pyridoimidazoles, pyridothiazoles, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,3-tirazolyl, 1,2,4-triazolyl, 1,2,5-triazolyl, 1,3,4-tirazolyl and xanthenyl.

Preference is given to pyridyl; such as 2-pyridyl, 3-pyridyl or 4-pyridyl; pyrrolyl; such as 2-pyrrolyl and 3-pyrrolyl; furyl; such as 2-furyl and 3-furyl; thienyl; such as 2-thienyl and 3-thienyl; imidazolyl; pyrazolyl, oxazolyl, isoxazolyl, thiazolyl, isothiazolyl, tetrazolyl, pyridazinyl, pyrazinyl, pyrimidinyl, indolyl, isoindolyl, benzofuranyl, benzothiophenyl, 1,3-benzodioxolyl, indazolyl, benzimidazolyl, benzoxazolyl, benzothiazolyl, quinolinyl, isoquinolinyl, chromanyl, isochromanyl, cinnolinyl, quinazolinyl, quinoxalinyl, phthalazinyl, pyridoimidazolyl, pyridopyridinyl, pyridopyrimidinyl, purinyl and pteridinyl.

Particular preference is given to a Het from the group aziridine, oxirane, azetidine, pyrrole, furan, thiophene, dioxole, imidazole, pyrazole, oxazole, isoxazole, thiazole, isothiazole, 1,2,3-triazole, 1,2,4-triazole, pyridine, pyran, thiopyran, pyridazine, pyrimidine, pyrazine, 1,4-dioxin, 1,2-oxazine, 1,3-oxazine, 1,4-oxazine, 1,2-thiazine, 1,3-thiazine, 1,4-thiazine, 1,2,3-triazine, 1,2,4-triazine, 1,3,5-triazine, azepine, 1,2-diazepine, 1,3-diazepine, 1,4-diazepine, indole, isoindole, benzofuran, benzothiophene, 1,3-benzodioxole, benzo[1,4]dioxin, 4H-benzo[1,4]oxazine, indazole, benzimidazole, benzoxazole, benzothiazole, quinoline, isoquinoline, chroman, isochroman, cinnoline, quinazoline, quinoxaline, phthalazine, pyridoimidazoles, pyridopyridines or pyridopyrimidines, etc. and also ring systems which ensue from the listed heterocycles by linking to or annelation with a carbocyclic ring, for Example benzoanelated, cycopentaanelated, cycohexaanelated or cycloheptaanelated derivatives of these heterocycles. Suitable nitrogen heterocycles can also be present as N oxides or as quaternary salts in which a suitable nitrogen atom is alkylated with —(C₁-C₄)-alkyl radicals. The Het groups can be unsubstituted or substituted in accordance with the listed definitions.

The term “azaheterocyclyl” 5-, 6- or 7-membered saturated carbocyclic ring wherein one ring carbon thereof is replaced by a nitrogen atom and has other ring carbon atoms thereof optionally replaced by a heteroatom that is an oxygen, sulfur or nitrogen atom.

The term “osteoarthrosis” means a disease which chiefly develops in connection with a disparity between the strain on and the load capacity of the individual joint components and joint tissues, which is associated with increasing destruction of the cartilage and which is in the main not inflammatory. Damage to the joint cartilage, such as fraying, demedullation and hyalinization, followed by reactive changes in the subchondral bone, and also capsule changes, is prominent in the pathology. The term “spondylosis” means an arthrosis of the vertebral bodies, with this arthrosis being characterized by a noninflammatory loss of cartilage from the vertebral bodies and intervertebral disks.

### EMBODIMENTS

Another embodiment of the invention relates to the compound of formula I wherein
R² is
   —(C₁-C₄)-alkyl, where alkyl is substituted, once, twice or three times, by
      —C(O)—O—R⁸,
      —(C₁-C₄)-alkyl-O—R⁸,
      phenyl that is substituted, once, twice or three times, independently of each other, by R¹¹, or
      Het that is azepine, azetidine, aziridine, benzimidazole, benzo[1,4]dioxin, 1,3-benzodioxole, benzofuran, 4H-benzo[1,4]oxazine, benzoxazole, benzothiazole, benzothiophene, quinazoline, quinoline, quinoxaline, chroman, cinnoline, oxirane, 1,2-diazepine, 1,3-diazepine, 1,4-diazepine, 1,4-dioxin, dioxole, furan, imidazole, indazole, indole, isoquinoline, isochroman, isoindole, isoxazole, isothiazole, 1,2-oxazine, 1,3-oxazine, 1,4-oxazine, oxazole, phthalazine, piperidine, pyran, pyrazine, pyrazole, pyridazine, pyridine, pyrimidine, pyridoimidazole, pyridopyridine, pyridopyrimidine, pyrrol, tetrazole, 1,2-thiazine, 1,3-thiazine, 1,4-thiazine, thiazole, thiophene, thiopyran, 1,2,3-triazine, 1,2,4-triazine, 1,3,5-triazine, 1,2,3-triazole or 1,2,4-triazole, and Het is unsubstituted or substituted, once, twice or three times, independently of each other, by R¹³
R³, R⁴, R⁵, R⁶ and R⁷ are identical or different and are
   hydrogen atom,
   halogen,
   —(C₁-C₆)-alkyl, in which alkyl is unsubstituted or substituted, once, twice or three times, by halogen, or
   —O—(C₁-C₆)-alkyl, in which alkyl is unsubstituted or substituted, once, twice or three times, by halogen,
R⁸ is
   hydrogen atom, or
   —(C₁-C₄)-alkyl,
R¹¹ is
   —(C₂-C₄)-alkyl-C(O)—O—R⁸,
   —O—(C₁-C₄)-alkyl-C(O)—O—R⁸,
   —N R¹⁴R¹⁵, wherein R¹⁴ and R¹⁵ taken together with the nitrogen atom to which they are attached form imidazolidine, isothiazolidine, isoxazolidine, morpholine, piperazine, piperidine, pyrazine, pyrazolidine, pyrrolidine, tetrazine or thiomorpholine, and wherein each nitrogen atom thereof is optionally independently unsubstituted or substituted by (C₁-C₄)-alkyl,
   —(CH₂)ₖ—N R⁹R¹⁰,
   —O—(C₂-C₄)-alkyl-NR⁹R₁₀, or
   —NH—C(O)—(C₁-C₄)-alkyl, wherein the alkyl is unsubstituted or substituted, once, twice or three times, by R¹²,
R⁹ and R¹⁰ are identical or different and are, independently of each other,
   hydrogen atom, or
   —(C₁-C₄)-alkyl, or
   taken together with the nitrogen atom to which they are attached form imidazolidine, isothiazolidine, isoxazolidine, morpholine, piperazine, piperidine, pyrazine, pyrazolidine, pyrrolidine, tetrazine or thiomorpholine, and wherein the nitrogen atom is optionally unsubstituted or substituted by —(C₁-C₄)-alkyl,
k is
   2, 3 or 4, and
R¹³ is
   halogen,
   amino,
   —C(O)—O—(C₁-C₄)-alkyl,
   —C(O)—OH,
   —(C₁-C₆)-alkyl that is unsubstituted or substituted, once, twice or three times, by halogen,
   —O—(C₁-C₆)-alkyl, wherein the alkyl is unsubstituted or substituted, once, twice or three times, by halogen,
   pyridyl, or
   phenyl that is unsubstituted or substituted, once or more than once and independently of each other, by a radical from the series halogen,
      —(C₁-C₄)-alkoxy and —(C₁-C₄)-alkyl.

Another embodiment of the invention relates to the compound of formula I wherein
R¹ is
   hydrogen,
R² is
   —(C₁-C₂)-alkyl that is substituted, once, twice or three times, by
      phenyl that is substituted, once, twice or three times, independently of each other, by R¹¹, or
      Het that is furan, imidazole, isothiazole, isoxazole, oxazole, pyrazole, pyridazine, pyridine, pyrimidine, pyrrole, thiazole, thiophene, 1,2,3-triazole or 1,2,4-triazole, and Het is unsubstituted or substituted, once, twice or three times, independently of each other, by R¹³,
R³, R⁴, R⁵, R⁶ and R⁷ are identical or different and are, independently of each other,
   hydrogen,
   halogen,
   methyl,
   trifluoromethyl,
   methoxy, or
   trifluoromethoxy,
R⁸ is
   hydrogen atom, or
   —(C₁-C₄)-alkyl,
R¹¹ is
   —(C₂-C₄)-alkyl-C(O)—O—R⁸,
   —O—(C₁-C₄)-alkyl-C(O)—O—R⁸,
   —N R¹⁴R¹⁵, wherein R¹⁴ and R¹⁵ taken together with the nitrogen atom to which they are attached form imidazolidine, isothiazolidine, isoxazolidine, morpholine, piperazine, piperidine, pyrazine, pyrazolidine, pyrrolidine, tetrazine or thiomorpholine, and wherein each nitrogen atom thereof is optionally independently unsubstituted or substituted by (C₁-C₄)-alkyl,
   —(CH₂)ₖ—N R⁹R¹⁰,
   —O—(C₂-C₄)-alkyl-NR⁹R¹⁰, or
   —NH—C(O)—(C₁-C₄)-alkyl, wherein the alkyl is unsubstituted or substituted, once, twice or three times, by R¹²,
R⁹ and R¹⁰ are identical or different and are, independently of each other,
   hydrogen atom, or
   —(C₁-C₄)-alkyl, or
   taken together with the nitrogen atom to which they are attached form imidazolidine, isothiazolidine, isoxazolidine, morpholine, piperazine, piperidine, pyrazine, pyrazolidine, pyrrolidine, tetrazine or thiomorpholine, and wherein the nitrogen atom is optionally unsubstituted or substituted by —(C₁-C₄)-alkyl,
k is
   2, 3 or 4,
R¹² is
   halogen,
   —C(O)—O—(C₁-C₄)-alkyl, or
   —C(O)—OH, and
R¹³ is
   halogen,
   amino,
   —C(O)—O—(C₁-C₄)-alkyl,
   —C(O)—OH,
   —(C₁-C₄)-alkyl that is unsubstituted or substituted, once, twice or three times, by halogen,
   —O—(C₁-C₄)-alkyl, wherein the alkyl is unsubstituted or substituted, once, twice or three times, by halogen,
   pyridyl, or
   phenyl that is unsubstituted or substituted, once or more than once and independently of each other, by a radical from the series halogen, —(C₁-C₄)-alkoxy and —(C₁-C₄)-alkyl.

Another particular embodiment of the invention is a process for the preparation of the compound according to formula I, comprising
a) reacting a compound of formula II 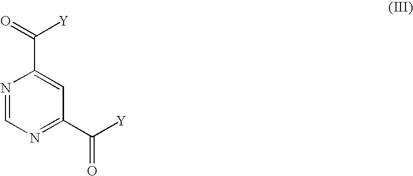
   wherein Y is
halogen, hydroxyl or C₁-C₄-alkoxy, or forms, together with the carbonyl group, an active ester or a mixed anhydride,
   with a compound of formula IIIa 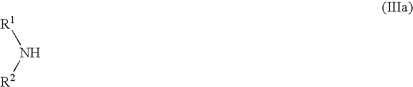
   wherein R¹ and R², have the meanings given in the compound of formula I, to form a compound of formula IVa 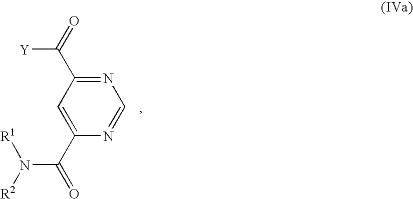
b) reacting the compound of formula IVa with a compound of formula IIIb 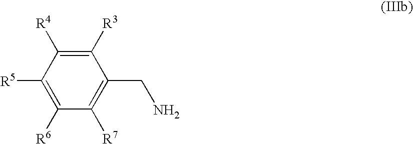
   wherein R³, R⁴, R⁵, R⁶ and R⁷ have the meanings given in the compound of formula I, to form the compound of formula I.

Another aspect of the aforesaid preparative method optionally includes purifying the compound of formula IVa, where appropriate, before reacting it according to step b).

Another particular embodiment of the invention is a process for the preparation of the compound according to formula I, comprising
a) reacting a compound of formula II 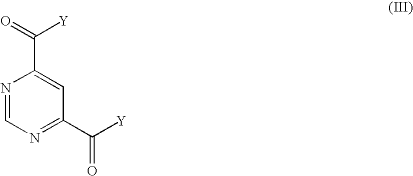
   wherein Y is
halogen, hydroxyl or C₁-C₄-alkoxy, or forms, together with the carbonyl group, an active ester or a mixed anhydride,
   with a compound of formula IIIb 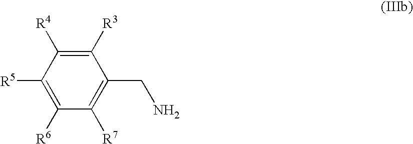
   wherein R³, R⁴, R⁵, R⁶ and R⁷ have the meanings given in the compound of formula I,
   to form a compound of formula IVb 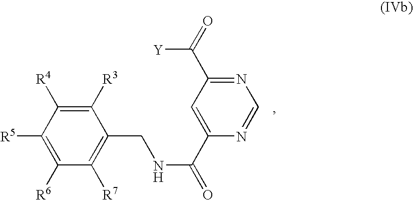
b) reacting the compound of formula IVb with a compound of formula IIIa 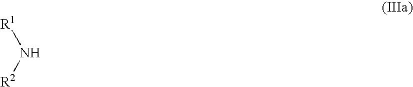
wherein R¹ and R², have the meanings given in the compound of formula I, to form the compound of formula I.

Another aspect of the aforesaid preparative method optionally includes purifying the compound of formula IVb, where appropriate, before reacting it according to step b).

Another aspect of the aforesaid preparative methods optionally includes converting the compound of formula I, prepared according to steps b) into a corresponding physiologically tolerated salt thereof

The preparation of a compound of formula I, and the preparation of the starting compounds and reagents that are required for this purpose are commercially available, can be prepared using known methods such as described in the literature or are prepared as described in more detail below.

The compound according to the invention is most easily prepared by mixing the two components, i.e., the pyrimidine derivative of the formula II and the amine of the formula IIIa or IIIb in equimolar quantities and reacting them, at temperatures of from −30° C. to 150° C., preferably at from 20° C. to 100° C., to give a compound of formula IVa or IVb and then reacting the compounds of the formula IVa or IVb with up to an equimolar quantity of the amine of the formula IIIb or IIIa in an analogous manner. The termination of the reaction can be determined, for example, by means of thin layer chromatography or HPLC-MS. A variant of this method is that the reaction is carried out in a suitable solvent, such as diethyl ether, dimethoxyethane or tetrahydrofuran, chlorinated hydrocarbons, such as methylene chloride, chloroform, trichloroethylene or tetrachloroethylene, benzene or toluene, or else polar solvents, such as dimethylformamide, acetone or dimethyl sulfoxide. The reaction temperatures in this connection are between room temperature and the boiling point of the solvent, with temperatures in the range from room temperature up to 130° C. being particularly preferred.

Reaction can also take place by way of a mixed anhydride, such as ethyl chloroformate, or by way of an active ester, such as para-nitrophenyl ester (Y═ClCH₂—COO— or NO₂—C₆H₄—O—).

A compound of formula II or a compound of formula IVa or IVb can also react with an amine of the formula IIIa or IIIb if Y is OH and the corresponding carboxylic acid is activated in situ using customary coupling reagents. Examples of these coupling reagents are carbodiimides, such as dicyclohexylcarbodiimide (DCC) or diisopropylcarbodiimide (DCI), or N,N′-carbonyldiazoles, such as N,N′-carbonyldiimidazole, or a uronium salt, such as O-((cyano(ethoxycarbonyl)methylene)amino)-1,1,3,3-tetramethyl-uroniumtetrafluoroborate (TOTU) or O-(7-azabenzotriazol-1-yl)-1,3,3,-tetramethyluronium hexafluorophosphate (HATU).

If amines of the formula IIIa or IIIb are not commercially available, they can be prepared from corresponding, commercially available starting compounds using methods known in the literature. Examples of suitable starting compounds for amines are nitriles, nitro compounds, carboxamides, carboxylic esters, carboxylic acids, aldehydes and bromides. Nitriles, nitro compounds and carboxamides can be reduced to amines using known methods. Carboxylic acids and carboxylic esters can be converted into the carboxamides. Aldehydes can be converted directly into the amines by means of a reductive amination with NH₄Ac/NaBH₄, or can be initially converted into the oximes using hydroxylamine and then being converted into the amines by reduction.

Where appropriate, the reaction can also take place in the presence of bases. Examples of suitable additional bases are carbonates or hydrogen carbonates, such as sodium carbonate or potassium carbonate or sodium hydrogen carbonate or potassium hydrogen carbonate, or tertiary amines, such as triethylamine, tributylamine or ethyldiisopropylamine, or heterocyclic amines, such as N-alkylmorpholine, pyridine, quinoline or dialkylanilines.

Where appropriate, the products, in particular the compound of formula IVa or IVb can be worked up, for example, by means of extraction or chromatography, e.g., through silica gel. The isolated product can be recrystallized and reacted, where appropriate, with a suitable acid to give a physiologically tolerated salt. Examples of suitable acids are: mineral acids, such as hydrochloric acid and hydrobromic acid, and also sulfuric acid, phosphoric acid, nitric acid or perchloric acid, or organic acids, such as formic acid, acetic acid, propionic acid, succinic acid, glycolic acid, lactic acid, malic acid, tartaric acid, citric acid, maleic acid, fumaric acid, phenylacetic acid, benzoic acid, methanesulfonic acid, toluenesulfonic acid, oxalic acid, 4-aminobenzoic acid, naphthalene-1,5-disulfonic acid or ascorbic acid.

Insofar as they are not commercially available, the starting compounds of the formula IIIa or IIIb can be prepared readily (e.g., Organikum, Organisch Chemisches grundpraktikum [Organicum, basic practical course in organic chemistry], 15th edn, VEB Deutscher Verlag der Wissenschaften, 1976; the methods index contains a review of the different possibilities on p. 822).

The starting compound of formula II is obtained, for example, by reacting pyrimidine-4,6-dicarboxylic acid, or pyridine-2,4-dicarboxylic acid, to give the corresponding pyrimidine-4,6-dicarbonyl halide or pyridine-2,4-dicarbonyl halide, preferably chloride (using methods known from the literature), preferably in the presence of a catalyst such as dimethylformamide. This acid halide can then be reacted, for example, either with a suitable alcohol, e.g., para-nitrobenzyl alcohol to give the corresponding active ester, or else with lower alcohols, such as methanol or ethanol, to give the corresponding esters. The pyrimidine-4,6-dicarboxylic acid can also initially be converted, in the added presence of a suitable carboxylic acid or of a carboxylic ester, such as ethyl chloroformate, into a mixed anhydride, which is then reacted with the amines of the compound of formulae IIIa or IIIb and IVa or IVb to give the products according to the invention. A corresponding method is also described in the literature.

The pyrimidine-4,6-dicarboxylic acid is prepared in accordance with methods known from the literature, for Example by oxidizing 4,6-dimethylpyrimidine, which, for its part, can be obtained, for example, by catalytically hydrogenating commercially available 2-mercapto-4,6-dimethylpyrimidine.

Insofar as compounds of the formula I permit diastereoisomeric or enantiomeric forms and accrue as their mixtures in the synthesis which is selected, separation into the pure stereoisomers is achieved either by chromatography on an optionally chiral support material or, provided the racemic compound of formula I is capable of salt formation, by fractionally crystallizing the diastereomeric salts which are formed using an optically active base or acid as auxiliary substance. Examples of suitable chiral stationary phases for the thin-layer or column chromatographic separation of enantiomers are modified silica gel supports (what are termed Pirkle phases) and also high molecular weight carbohydrates, such as triacetyl cellulose. Following appropriate derivatization, which is known to the skilled person, gas-chromatographic methods on chiral stationary phases can also be used for analytical purposes. In order to separate the enantiomers of the racemic carboxylic acids, the diastereomeric salts, which differ in solubility, are formed using an optically active, as a rule commercially available, base such as (−)-nicotine, (+)- and (−)-phenylethylamine, quinine bases, L-lysine or L- and D-arginine, the more sparingly soluble component is isolated as a solid, the more readily soluble diastereomer is separated out from the mother liquor, and the pure enantiomers are isolated from the diastereomer salts which have been obtained in this way. The racemic compounds of the formula I which contain a basic group, such as amino group, can, in what is in principle the same manner, be converted into the pure enantiomers using optically active acids, such as (+)-camphor-10-sulfonic acid, D- and L-tartaric acid, D- and L-lactic acid and (+) and (−)-mandelic acid. Chiral compounds which contain alcohol or amine functions can also be converted into the corresponding esters or amides using appropriately activated or optionally N-protected enantiomerically pure amino acids or, conversely, chiral carboxylic acids can be converted into the amides using carboxyl-protected enantiomerically pure amino acids or into the corresponding chiral esters using enantiomerically pure hydroxycarboxylic acids such as lactic acid. The chirality of the amino acid or alcohol radical which has been introduced in enantiomerically pure form can then be used for separating the isomers by the diastereomers, which are now present, being separated by means of crystallization or chromatography on suitable stationary phases and, after that, using suitable methods to once again eliminate the entrained chiral molecule moiety.

Acidic or basic products of the compound of formula I may be present in the form of their salts or in free form. Preference is given to pharmacologically tolerated salts, e.g., alkali metal salts or alkaline earth metal salts or hydrochlorides, hydrobromides, sulfates, hemisulfates, all possible phosphates and also salts of the amino acids, natural bases or carboxylic acids.

Physiologically tolerated salts are prepared in a manner known per se from compounds of the formula I, including their stereoisomeric forms, which are capable of salt formation. The carboxylic acids form stable alkali metal salts, alkaline earth metal salts or optionally substituted ammonium salts with basic reagents such as hydroxides, carbonates, hydrogencarbonates, alkoxides and ammonia or organic bases, for Example trimethylamine, triethylamine, ethanolamine or triethanolamine, or else basic amino acids, for Example lysine, ornithine or arginine. Insofar as the compounds of the formula I possess basic groups, stable acid addition salts can also be prepared using strong acids.

Both inorganic and organic acids, such as hydrochloric acid, hydrobromic acid, sulfuric acid, phosphoric acid, methanesulfonic acid, benzenesulfonic acid, p-toluenesulfonic acid, 4-bromobenzenesulfonic acid, cyclohexylamidosulfonic acid, trifluoromethylsulfonic acid, acetic acid, oxalic acid, tartaric acid, succinic acid and trifluoroacetic acid are suitable for this purpose.

As a result of their pharmacological properties, the compound of formula I is suitable for the prophylaxis and therapy of all those diseases whose course involves a physiologically detrimental increase in the activity of MMP 13.

These diseases include degenerative joint diseases, such as osteoarthroses, spondyloses and cartilage loss following joint trauma or relatively long joint immobilization following meniscus or patella injuries or ligament rupture. They also include diseases of the connective tissue, such as collagenoses, periodontal diseases, wound healing disturbances and chronic diseases of the locomotory apparatus such as inflammatory, immunologically determined or metabolism-determined, acute and chronic arthritides, arthropathies, myalgias and disturbances of bone metabolism, or cancers, such as breast cancer.

A pharmaceutical preparation according to the invention can be administered by subcutaneous, intraarticular, intraperitoneal or intravenous injection. Intraarticular injection is preferred. Rectal, oral, inhalative or transdermal administration is also possible.

The invention also relates to a pharmaceutical preparation comprising a pharmaceutically effective amount of at least one compound of formula I according to claim 1 and a pharmaceutically suitable and physiologically tolerated carrier, and optionally an appropriate other suitable active compound, additive or auxiliary substance.

The compound of formula I is mixed with the additives, such as carrier substances, stabilizers or inert diluents, which are suitable for the purpose and brought into suitable administration forms, such as tablets, sugar-coated tablets, hard gelatin capsules, aqueous, alcoholic or oily suspensions or aqueous or oily solutions, using the customary methods. Examples of inert carrier substances that can be used are gum arabic, magnesium oxide, magnesium carbonate, potassium phosphate, lactose, glucose or starch, in particular corn starch. In this connection, the preparation can be effected either as dry granules or as wet granules. Examples of suitable oily carrier substances or solvents are vegetable or animal oils, such as sunflower oil or cod liver oil.

For subcutaneous, intraarticular, intraperitoneal or intravenous administration, the active compounds are, if desired, brought into solution, suspension or emulsion using the substances that are suitable for this purpose, such as solubilizers, emulsifiers or other auxiliary substances. Examples of suitable solvents are physiological sodium chloride solution or alcohols, e.g., ethanol, propanol or glycerol, and, in addition, sugar solutions, such as solutions of glucose or mannitol, or else a mixture that is composed of the different solvents mentioned.

Customary adjuvants, such as carrier substances, disintegrants, binding agents, coating agents, swelling agents, glidants or lubricants, flavorings, sweeteners and solubilizers are also used. Auxiliary substances that are frequently employed and may be mentioned are magnesium carbonate, titanium dioxide, lactose, mannitol and other sugars, talc, milk protein, gelatin, starch, cellulose and its derivatives, animal and vegetable oils, such as cod liver oil, sunflower oil, peanut oil or sesame seed oil, polyethylene glycol and solvents such as sterile water and monohydric or polyhydric alcohols, such as glycerol.

The compound of formula I is preferably prepared as pharmaceutical preparations and administered in dosage units, with each unit containing a defined dose of the compound of formula I as the active constituent. For this purpose, the compound of formula I can be administered orally in doses of from 0.01 mg/kg/day to 25.0 mg/kg/day, preferably from 0.01 mg/kg/day to 5.0 mg/kg/day, or parenterally in doses of from 0.001 mg/kg/day to 5 mg/kg/day, preferably of from 0.001 mg/kg/day to 2.5 mg/kg/day. The dosage can also be increased in severe cases. However, smaller doses are also adequate in many cases. These figures relate to the treatment of an adult.

### EXAMPLES

The invention is explained in more detail below with the aid of examples.

### Example 1

### tert-Butyl [4-({[6-(4-fluoro-3-methylbenzylcarbamoyl)pyrimidine-4-carbonyl]amino}methyl)phenoxy]acetate

tert-Butyl (4-formylphenoxy)acetate: 10 g (0.0819 mol) of 4-hydroxybenzaldehyde and 15.97 g (0.0819 mol) of tert-butyl bromoacetate were dissolved in 200 ml of 2-butanone, after which 11.32 g (0.0819 mol) of potassium carbonate were added and the mixture was heated under reflux for 2 hours (h). The mixture was then concentrated under reduced pressure and the residue was taken up in water and the solution was extracted three times with dichloromethane. The organic phase was dried (MgSO₄), filtered and concentrated under reduced pressure.

Yield: 18.72 g (97%)

tert-Butyl [4-(hydroxyiminomethyl)phenoxy]acetate: 18.72 g (0.0792 mol) of tert-butyl (4-formylphenoxy)acetate were dissolved in 200 ml of water/ethanol (1:1), after which 6.056 g (0.0872 mol) of hydroxyammonium chloride and 3.169 g (0.0792 mol) of sodium hydroxide were added and the mixture was stirred under reflux for 2.5 hours (h). The mixture was concentrated under reduced pressure and the residue was taken up in water and the solution was extracted with ethyl acetate. The organic phase was dried (MgSO₄), filtered and concentrated under reduced pressure.

Yield: 19.9 g (100%) MS (ES+): m/e=251.12

tert-Butyl (4-aminomethylphenoxy)acetate: 6 g (0.0239 mol) of tert-butyl [4-(hydroxyiminomethyl)phenoxy]acetate were dissolved in 10 ml of water/ethanol (1:1) and hydrogenated, under a pressure of 5 bar and at room temperature, over 0.06 g of Pd/C (10%). After 3 hours (h), the mixture was filtered through Celite and concentrated under reduced pressure. Yield: 4.26 g (75%)

Methyl 6-(4-fluoro-3-methylbenzylcarbamoyl)pyrimidine-4-carboxylate: the compound was prepared in analogy with methyl 6-benzylcarbamoylpyrimidine-4-carboxylate.

6-(4-Fluoro-3-methylbenzylcarbamoyl)pyrimidine-4-carboxylic acid: 0.889 g (0.0222 mol) of sodium hydroxide in 6 ml of water was added to 8.75 g (0.0202 mol) of methyl 6-(4-fluoro-3-methylbenzylcarbamoyl)pyrimidine-4-carboxylate in 80 ml of methanol and the mixture was stirred at room temperature (RT). After 2.5 hours, the solvent was removed under reduced pressure and the residue was dissolved in 300 ml of water and the solution was acidified with concentrated HCl. The precipitate was filtered off and dried.

Yield: 5.43 g (95%) MS (ES+): m/e=289.09

tert-Butyl [4-({[6-(4-fluoro-3-methylbenzylcarbamoyl)pyrimidine-4-carbonyl]-amino}methyl)phenoxy]acetate: 150 mg (0.519 mmol) of 6-(4-fluoro-3-methlbenzylcarbamoyl)pyrimidine-4-carboxylic acid were dissolved in 5 ml of DMF, after which 170 mg (0.519 mmol) of TOTU were added and the mixture was stirred at RT for 30 minutes. 147.6 mg (0.62 mmol) of tert-butyl (4-aminomethylphenoxy)acetate and 119.55 mg (1.038 mmol) of N-ethylmorpholine were then added and the mixture was stirred at RT for 12 hours. The solvent was removed under reduced pressure and the residue was purified by preparative HPLC (water/acetonitrile). The homogeneous fractions were concentrated under reduced pressure and freeze-dried. Yield: 126 mg (48%) MS (ES−): m/e=508.21

### Example 2

### [4-({[6-(4-Fluoro-3-methylbenzylcarbamoyl)pyrimidine-4-carbonyl]-amino}methyl)phenoxy]acetic acid

83.6 mg (0.16 mmol) of tert-butyl [4-({[6-(4-fluoro-3-methylbenzylcarbamoyl-moyl)pyrimidine-4-carbonyl]amino}methyl)phenoxy]acetate (Example 31) were stirred at RT for 4 hours in 90% trifluoroacetic acid. Acetonitrile/water was then added and the precipitate was filtered off and dried.

Yield: 55 mg (76%) MS (ES+): m/e=452.15

The following compounds were prepared by a method analogous to that of Example 1.

TABLE 1

| Example | Structure | MS (ESI+) |
|---|---|---|
| 1 | 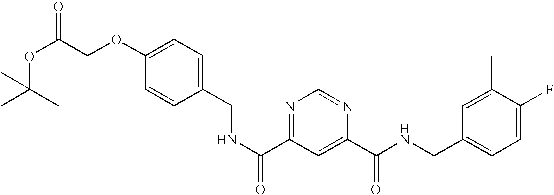 | 508.21 |
| 2 | 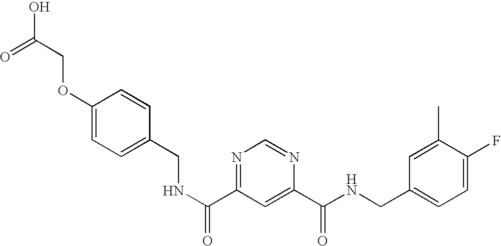 | 452.15 |
| 3 | 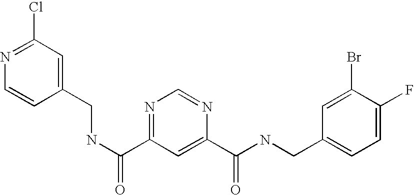 | 477.00 |
| 4 | 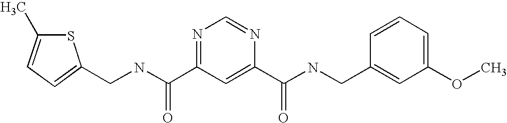 | 396.13 |
| 5 | 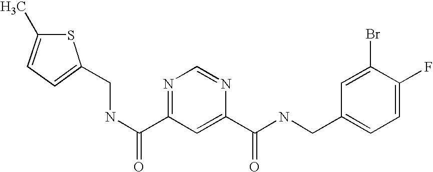 | 463.02 |
| 6 | 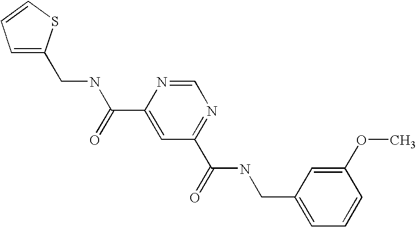 | 382.11 |
| 7 | 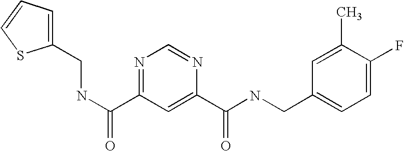 | 384.11 |
| 8 | 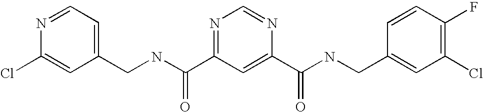 | 433.05 |
| 9 | 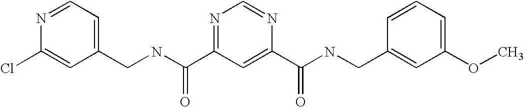 | 411.11 |
| 10 | 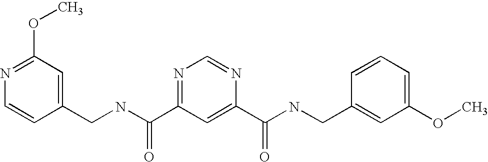 | 407.16 |
| 11 | 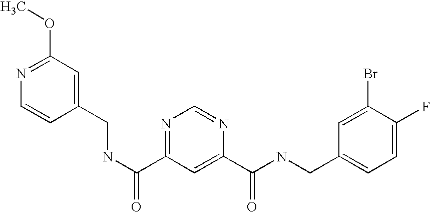 | 473.05 |
| 12 | 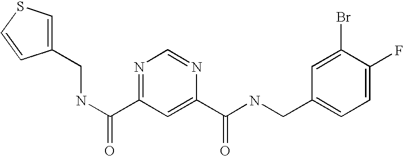 | 448.00 |
| 13 | 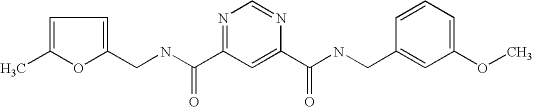 | 380.15 |
| 14 | 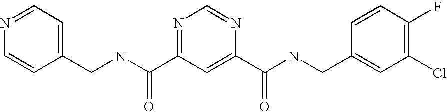 | 399.09 |
| 15 | 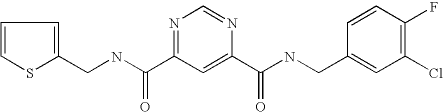 | 404.05 |
| 16 | 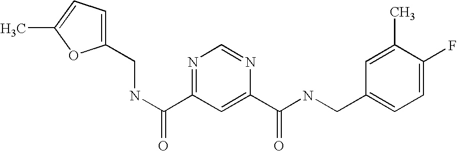 | 382.14 |
| 17 | 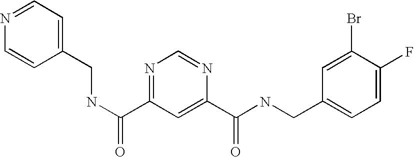 | 443.04 |
| 18 | 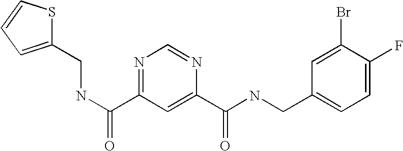 | 448.00 |
| 19 | 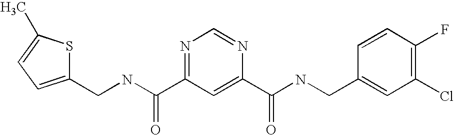 | 418.07 |
| 20 | 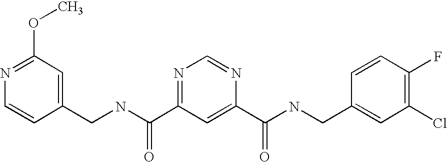 | 429.10 |
| 21 | 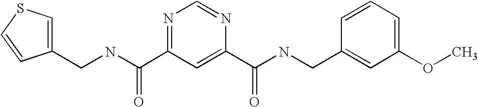 | 382.11 |
| 22 | 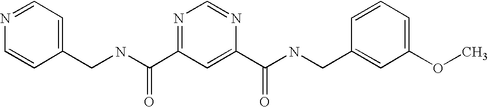 | 377.15 |
| 23 | 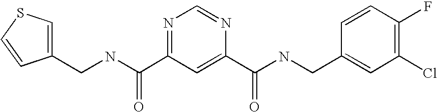 | 404.05 |
| 24 | 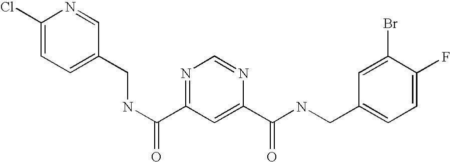 | 477.00 |
| 25 | 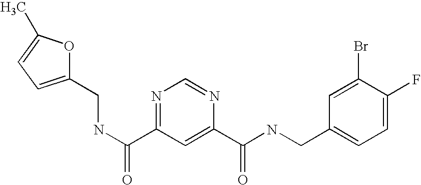 | 449.03 |
| 26 | 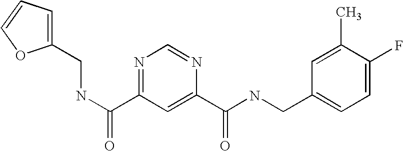 | 368.13 |
| 27 | 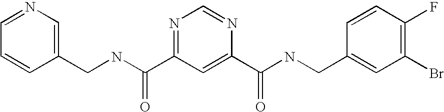 | 443.04 |
| 28 | 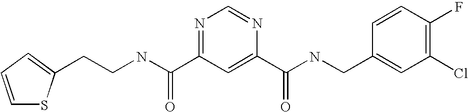 | 418.07 |
| 29 | 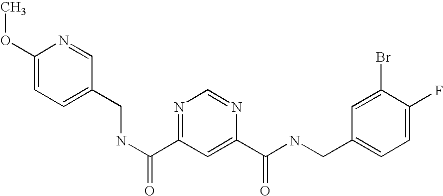 | 473.05 |
| 30 | 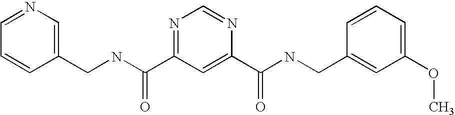 | 377.15 |
| 31 | 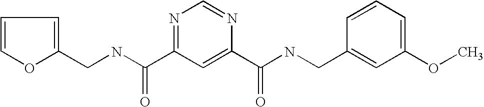 | 366.13 |
| 32 | 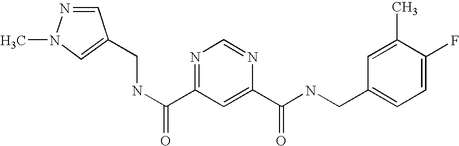 | 382.16 |
| 33 | 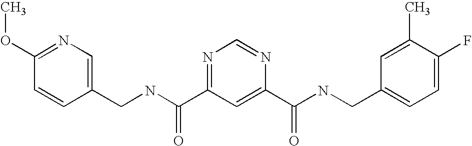 | 409.15 |
| 34 | 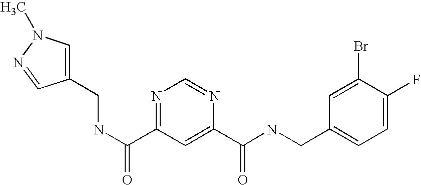 | 446.05 |
| 35 | 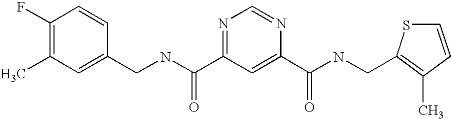 | 398.12 |
| 36 | 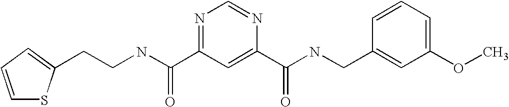 | 396.00 |
| 37 | 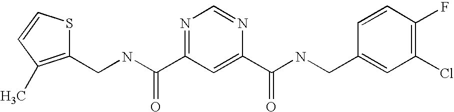 | 418.07 |
| 38 | 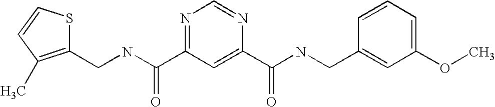 | 396.13 |
| 39 | 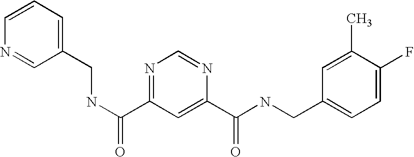 | 379.14 |
| 40 | 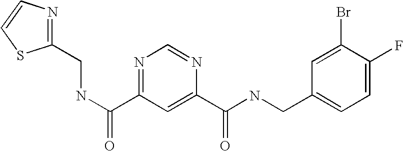 | 449.00 |
| 41 | 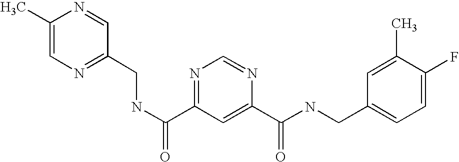 | 394.16 |
| 42 | 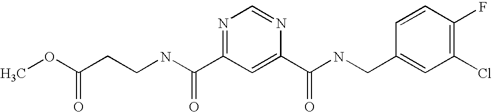 | 394.08 |
| 43 | 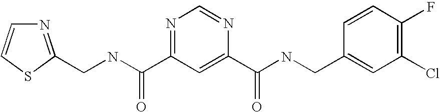 | 405.05 |
| 44 | 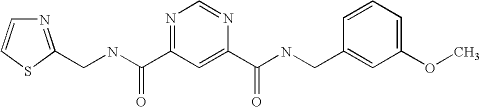 | 383.00 |
| 45 | 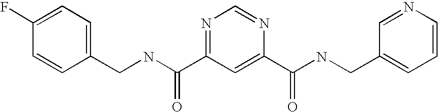 | 365.13 |
| 46 | 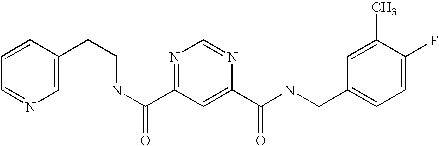 | 393.16 |
| 47 | 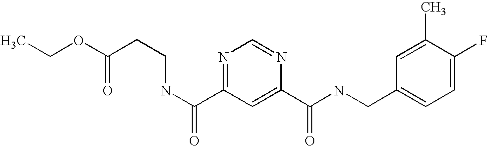 | 388.15 |
| 48 | 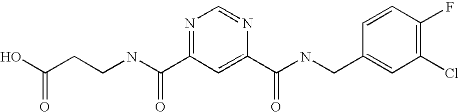 | 380.07 |
| 49 | 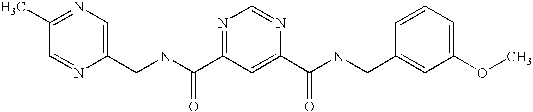 | 392.16 |
| 50 | 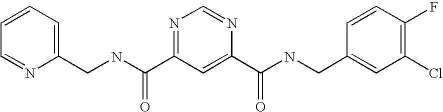 | 399.09 |
| 51 | 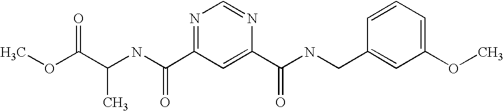 | 434.16 |
| 52 | 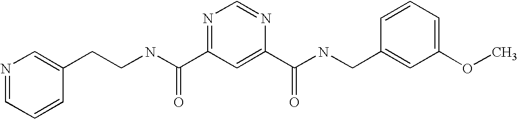 | 391.16 |
| 53 | 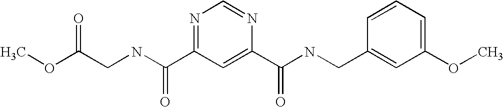 | 358.13 |
| 54 | 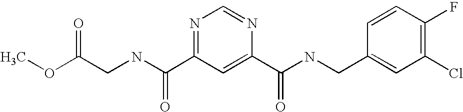 | 380.07 |
| 55 | 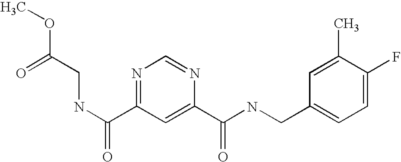 | 360.12 |
| 56 | 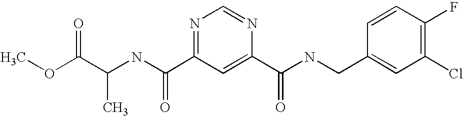 | 394.08 |
| 57 | 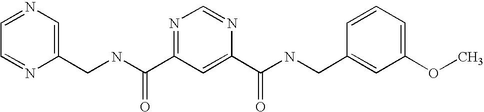 | 392.16 |
| 58 | 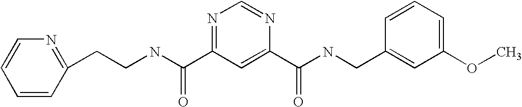 | 391.16 |
| 59 | 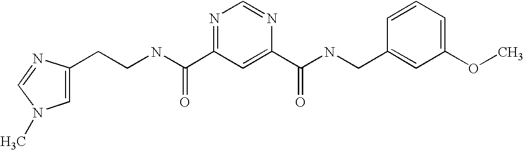 | 408.15 |
| 60 | 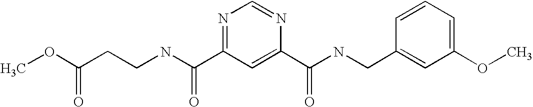 | 372.14 |
| 61 | 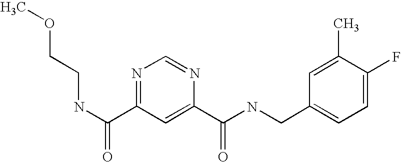 | 346.14 |
| 62 | 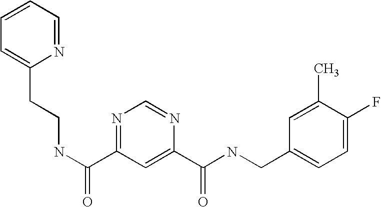 | 393.00 |
| 63 | 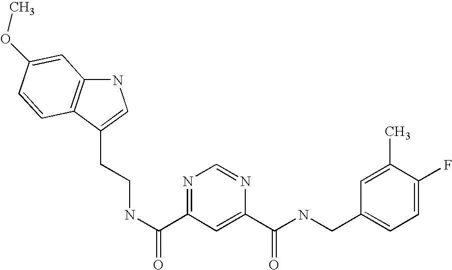 | 461.00 |
| 64 | 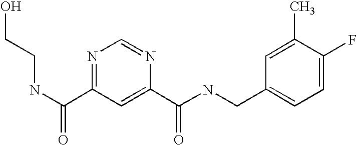 | 332.13 |
| 65 | 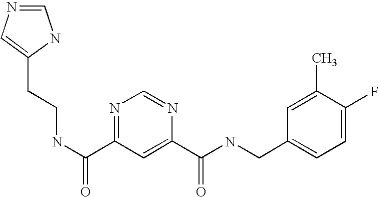 | 382.16 |
| 66 | 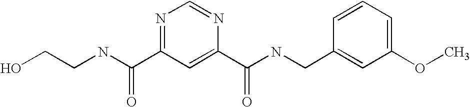 | 330.13 |
| 67 | 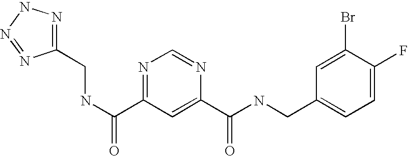 | 434.03 |
| 69 | 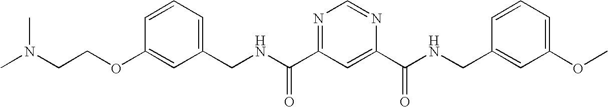 | 463.22 |
| 70 | 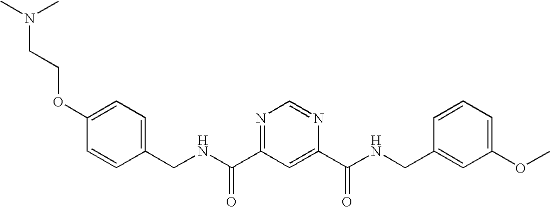 | 463.00 |
| 71 | 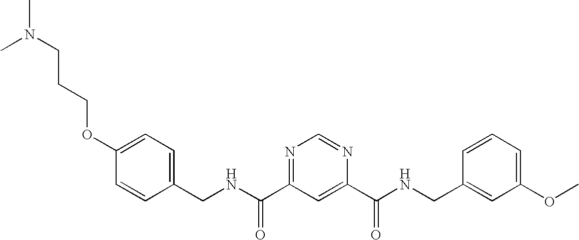 | 477.24 |
| 72 | 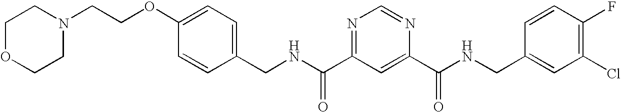 | 527.17 |
| 73 | 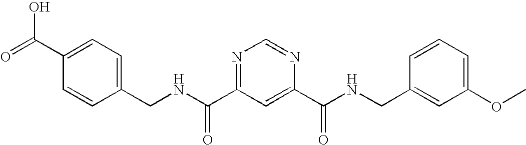 | 420.14 |
| 74 | 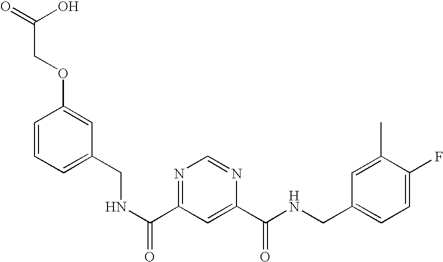 | 452.15 |
| 75 | 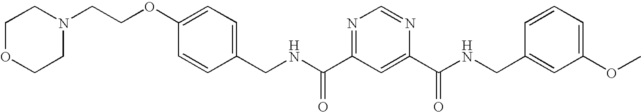 | 505.23 |
| 76 | 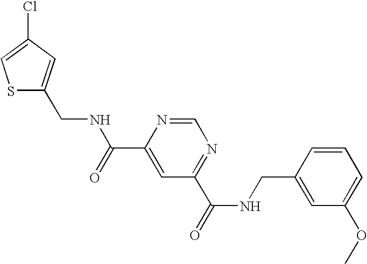 | 416.07 |
| 77 | 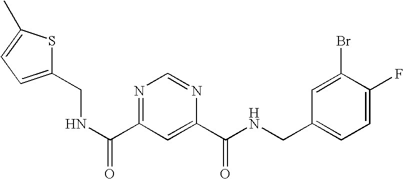 | 465.05 |
| 78 | 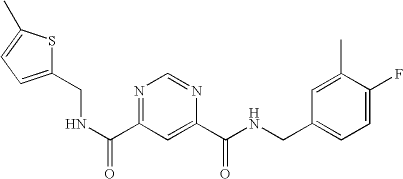 | 399.22 |
| 79 | 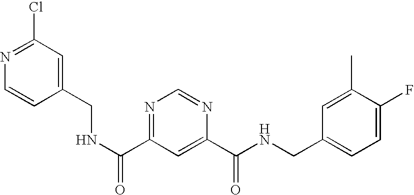 | 413.11 |
| 80 | 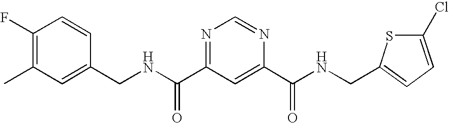 | 418.07 |
| 81 | 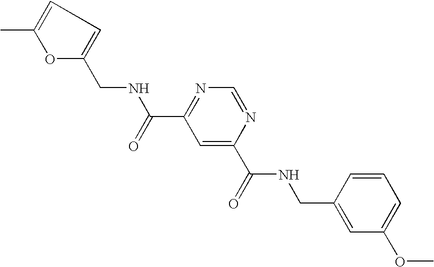 | 380.15 |
| 82 | 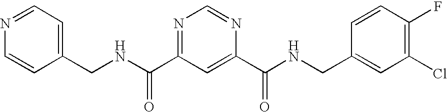 | 399.09 |
| 83 | 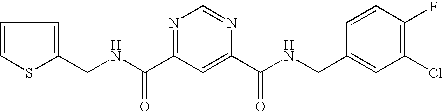 | 404.05 |
| 84 | 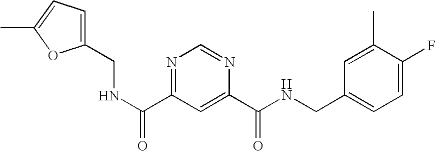 | 382.14 |
| 85 | 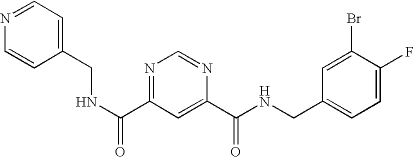 | 443.04 |
| 86 | 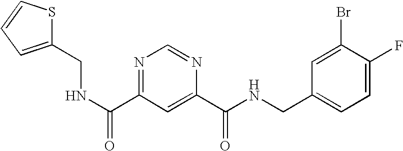 | 448.00 |
| 87 | 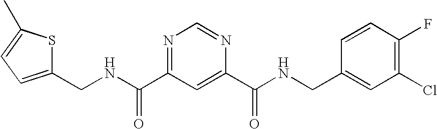 | 418.07 |
| 88 | 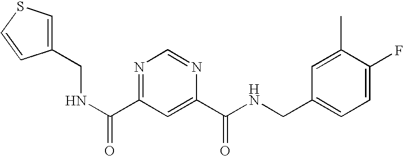 | 384.11 |
| 89 | 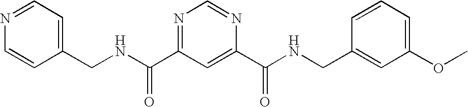 | 377.15 |
| 90 | 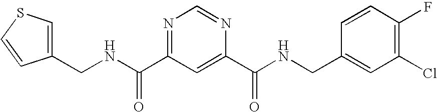 | 404.05 |
| 91 | 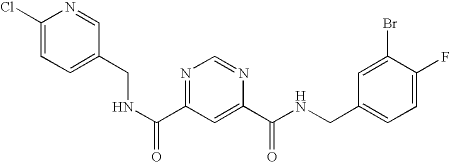 | 477.00 |
| 92 | 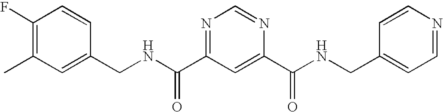 | 379.14 |
| 93 | 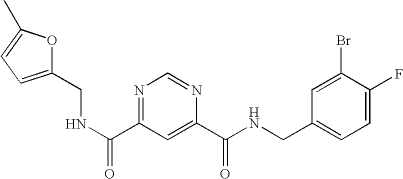 | 449.03 |

### Pharmacological Examples

Determining the enzyme activity of the catalytic domain of human collagenase 3 (MMP-13).

This protein is obtained as an inactive proenzyme from INVITEK, Berlin (Catalog No. 30 100 803). Activating the proenzyme:

2 parts by volume of proenzyme are incubated with 1 part by volume of APMA solution at 37° C. for 1.5 hours. The APMA solution is prepared from a 10 mmol/l solution of p-aminophenylmercuric acetate in 0.1 mmol/l NaOH by diluting with 3 parts by volume of tris/HCl buffer, pH 7.5 (see below). The pH is adjusted to between 7.0 and 7.5 by adding 1 mmol/I HCl. After the enzyme has been activated, it is diluted to a concentration of 1.67 μg/ml using the tris/HCl buffer.

In order to measure the enzyme activity, 10 μl of enzyme solution are incubated for 15 minutes with 10 μl of a 3% (v/v) buffered solution of dimethyl sulfoxide (reaction 1). In order to measure the enzyme inhibitor activity, 10 μl of enzyme solution are incubated with 10 μl of a 3% (v/v) buffered solution of dimethyl sulfoxide containing the enzyme inhibitor (reaction 2).

In the case of both reaction 1 and reaction 2, the enzyme reaction is monitored by fluorescence spectroscopy (328 nm (extinction)/393 nm (emission)) after adding 10 μl of a 3% (v/v) aqueous solution of dimethyl sulfoxide containing 0.75 mmol of the substrate/l.

The enzyme activity is depicted as increase in extinction/minute.

The effect of the inhibitor is calculated as a percentage inhibition using the following formula:
% inhibition=100−[(increase in extinction/minute in reaction 2)/(increase in extinction/minute in reaction 1)×100].

The IC₅₀, i.e., the inhibitor concentration that is required for inhibiting the enzyme activity by 50%, is determined graphically by plotting the percentage inhibitions at different inhibitor concentrations.

The buffer solution contains 0.05% Brij (Sigma, Deisenhofen, Germany) and 0.1 mol of tris/HCl/l, 0.1 mol of NaCl/l and 0.01 mol of CaCl₂/l (pH=7.5). The enzyme solution contains 1.67 μg of the enzyme domain/ml. The substrate solution contains 0.75 mmol/l of the fluorogenic substrate (7-methoxycoumarin4-yl)acetyl-Pro-Leu-Gly-Leu-3-(2′,4′-dinitrophenyl)-L-2,3-diaminopropionyl-Ala-Arg-NH₂ (Bachem, Heidelberg, Germany).

Table 2 below shows the results.

TABLE 2

| Example | IC₅₀ MMP13 (nM) |
|---|---|
| 1 | 12 |
| 2 | 7 |
| 4 | 24 |
| 11 | 30 |
| 20 | 50 |
| 28 | 100 |
| 35 | 200 |
| 44 | 500 |

Determining the enzyme activity of the catalytic domain of human neutrophil collagenase (MMP-8) and of human Stromelysin (MMP-3).

The enzymes human neutrophil collagenase and human Stromelysin, prepared as active catalytic domains, were tested as described in Weithmann et al Inflamm Res, 46 (1997), pages 246-252. The measurement of the enzyme activity, and the determination of the inhibitory effect of inhibitors on the enzyme activity, were also carried out as described in that publication.

When determining human neutrophil collagenase and human stromelysin, the compounds described in the above examples in each case had IC₅₀ values of more than 100,000 nm. These compounds are thus virtually without activity as regards inhibiting MMP 3 and 8.

Determining prolyl hydroxylase inhibition by the method of Majamaa Eur. J. Biochem. 138 (1984) 239-245 using the version of Kaule and guenzler Analytical Biochemistry 184 (1990) 291-297.

0.03 μg of prolyl 4-hydroxylase, which was prepared, by the method of Kedersa, Collagen Relat. Res. 1 (1981) 345-353, from homogenized 14-day-old chick embryos by ammonium sulfate precipitation and affinity chromatography on poly-L-proline-coupled Sepharose 4B and subsequent DEAE cellulose chromatography, was incubated, at 37° C. for one hour, in 0.05 ml of an 0.04 M tris-HCl solution (pH=7.5) containing 0.05 mM FeSO₄, 0.1 mM 2-oxo-[5-C-14]glutarate (100,000 dpm), 20 μg (0.4 mg/ml) of (Pro-Pro-Gly)₁₀ (Protein Research Foundation, Minoh, Osaka, Japan), 1 mM ascorbic acid, 0.4 mg of catalase/ml, 0.5 mM dithiothreitol and 2 mg of bovine serum albumin/ml, as well as the concentration of the inhibitor to be measured (the comparison experiment was carried out without any inhibitor). After that, 25 μl of a 20 mM succinate and 2-oxaglutarate solution, and also 25 μl of a 0.16 M 2,4-DNPH solution containing 30% HClO₄, were added. After the mixture had been incubated at room temperature for a further 30 minutes, it was centrifuged at 3000 g for 5 minutes. The radioactivity of 100 μl of the supernatant was determined by liquid scintillation. Production of the [1-C-14] succinate was given in dpm. Unless otherwise indicated, all the chemicals and reagents were obtained from Sigma, Sigma D-82024 Taufkirchen.

The compounds tested in Table 3 were used at concentrations of from 0.1 μM to 1 mM. Where it was possible, the IC₅₀ value was determined by graphic extrapolarization of the individual results. This value denotes the concentration of active inhibitors that led to 50% inhibition of the enzyme.

TABLE 3

| | Prolyl hydroxylase |
|---|---|
| Example | IC₅₀ (μM) |
| Pyrimidine-4,6- | 1.2 |
| dicarboxylic acid | |
| 7 | No inhibition |
| 14 | No inhibition |
| 16 | No inhibition |
| 22 | No inhibition |
| 69 | No inhibition |
| 70 | No inhibition |
| 71 | No inhibition |
| 72 | No inhibition |
| 83 | No inhibition |
| 84 | No inhibition |

The present invention may be embodied in other specific forms without departing from the spirit or essential attributes thereof.

1. A compound of formula I 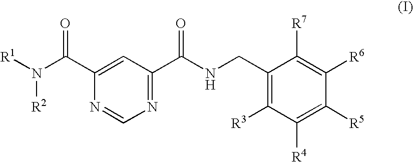
wherein
R¹ is
hydrogen atom or —(C₁-C₆)-alkyl,
R² is
—(C₁-C₆)-alkyl that is substituted, once, twice or three times, by
—C(O)—O—R⁸,
—(C₁-C₆)-alkyl-O—R⁸,
—(C₆-C₁₄)-aryl that is substituted, once, twice or three times, independently of each other, by R¹¹ or
Het that is a saturated or unsaturated monocyclic or bicyclic, 3- to 10-membered heterocyclic ring system which contains 1, 2 or 3 identical or different ring heteroatoms selected from the group consisting of nitrogen, oxygen and sulfur and is unsubstituted or substituted, once or more than once, by R¹³,
R³, R⁴, R⁵, R⁶ and R⁷ are identical or different and are, independently of each other,
hydrogen
halogen,
—(C₁-C₆)-alkyl, in which alkyl is unsubstituted or substituted, once, twice or three times, by halogen,
—O—(C₁-C₆)-alkyl, in which alkyl is unsubstituted or substituted, once, twice or three times, by halogen, or
—S—(C₁-C₆)-alkyl,
R⁸ is
hydrogen atom, or
—(C₁-C₆)-alkyl,
R¹¹ is
—(C₂-C₆)-alkyl-C(O)—O—R⁸,
—O—(C₁-C₆)-alkyl-C(O)—O—R⁸,
—NR¹⁴R¹⁵,
—(CH₂)ₖ—NR⁹R¹⁰,
—O—(C₂-C₆)-alkyl-NR⁹R¹⁰, or
—NR⁸—C(O)—(C₁-C₆)-alkyl, in which alkyl is unsubstituted or substituted, once, twice or three times, by R¹²,
R⁹ and R¹⁰ are identical or different and are, independently of each other,
hydrogen atom, or
—(C₁-C₆)-alkyl, or
taken together with the nitrogen atom to which they are attached form a 5-, 6- or 7-membered saturated azaheterocyclyl ring wherein one or two further carbon atoms thereof are optionally replaced by a heteroatom that is an oxygen, sulfur or nitrogen atom, and wherein the nitrogen atom is optionally unsubstituted or substituted by (C₁-C₆)-alkyl,
k is
2, 3, 4 or 5,
R¹² is
halogen,
cyano,
nitro,
hydroxyl,
amino,
—C(O)—O—(C₁-C₆)-alkyl, or
—C(O)—OH,
R¹³ is
halogen,
cyano,
nitro,
hydroxyl,
amino,
—C(O)—O—(C₁-C₆)-alkyl,
—C(O)—OH,
—(C₁-C₆)-alkyl that is unsubstituted or substituted, once, twice or three times, by halogen,
—O—(C₁-C₆)-alkyl, where alkyl is unsubstituted or substituted, once, twice or three times, by halogen,
pyridyl, or
phenyl that is unsubstituted or substituted, once or more than once and independently of each other, by a radical from the series halogen, (C₁-C₆)-alkoxy and (C₁-C₆)-alkyl, and
R¹⁴ and R¹⁵ together with the nitrogen atom to which they are attached form a 5-, 6- or 7-membered saturated azaheterocyclyl ring wherein one or two further carbon atoms thereof are optionally replaced by a heteroatom that is oxygen, sulfur or nitrogen, and wherein each nitrogen atom thereof is optionally independently unsubstituted or substituted by (C₁-C₆)-alkyl, or
stereoisomer thereof, a mixture of stereoisomers thereof in any ratio, or physiologically tolerable salt thereof.

2. The compound according to claim 1, wherein
R² is
—(C₁-C₄)-alkyl, where alkyl is substituted, once, twice or three times, by
—C(O)—O—R⁸,
—(C₁-C₄)-alkyl-O—R⁸,
phenyl that is substituted, once, twice or three times, independently of each other, by R¹¹, or
Het that is azepine, azetidine, aziridine, benzimidazole, benzo[1,4]dioxin, 1,3-benzodioxole, benzofuran, 4H-benzo[1,4]oxazine, benzoxazole, benzothiazole, benzothiophene, quinazoline, quinoline, quinoxaline, chroman, cinnoline, oxirane, 1,2-diazepine, 1,3-diazepine, 1,4-diazepine, 1,4-dioxin, dioxole, furan, imidazole, indazole, indole, isoquinoline, isochroman, isoindole, isoxazole, isothiazole, 1,2-oxazine, 1,3-oxazine, 1,4-oxazine, oxazole, phthalazine, piperidine, pyran, pyrazine, pyrazole, pyridazine, pyridine, pyrimidine, pyridoimidazole, pyridopyridine, pyridopyrimidine, pyrrol, tetrazole, 1,2-thiazine, 1,3-thiazine, 1,4-thiazine, thiazole, thiophene, thiopyran, 1,2,3-triazine, 1,2,4-triazine, 1,3,5-triazine, 1,2,3-triazole or 1,2,4-triazole, and Het is unsubstituted or substituted, once, twice or three times, independently of each other, by R¹³
R³, R⁴, R⁵, R⁶ and R⁷ are identical or different and are
hydrogen atom,
halogen,
—(C₁-C₆)-alkyl, in which alkyl is unsubstituted or substituted, once, twice or three times, by halogen, or
—O—(C₁-C₆)-alkyl, in which alkyl is unsubstituted or substituted, once, twice or three times, by halogen,
R⁸ is
hydrogen atom, or
—(C₁-C₄)-alkyl,
R¹¹ is
—(C₂-C₄)-alkyl-C(O)—O—R⁸,
—O—(C₁-C₄)-alkyl-C(O)—O—R⁸,
—N R¹⁴R¹⁵, wherein R¹⁴ and R¹⁵ taken together with the nitrogen atom to which they are attached form imidazolidine, isothiazolidine, isoxazolidine, morpholine, piperazine, piperidine, pyrazine, pyrazolidine, pyrrolidine, tetrazine or thiomorpholine, and wherein each nitrogen atom thereof is optionally independently unsubstituted or substituted by (C₁-C₄)-alkyl,
—(CH₂)ₖ—N R⁹R¹⁰,
—O—(C₂-C₄)-alkyl-NR⁹R¹⁰, or
—NH—C(O)—(C₁-C₄)-alkyl, wherein the alkyl is unsubstituted or substituted, once, twice or three times, by R¹²,
R⁹ and R¹⁰ are identical or different and are, independently of each other,
hydrogen atom, or
—(C₁-C₄)-alkyl, or
taken together with the nitrogen atom to which they are attached form imidazolidine, isothiazolidine, isoxazolidine, morpholine, piperazine, piperidine, pyrazine, pyrazolidine, pyrrolidine, tetrazine or thiomorpholine, and wherein the nitrogen atom is optionally unsubstituted or substituted by —(C₁-C₄)-alkyl,
k is
2, 3 or 4, and
R¹³ is
halogen,
amino,
—C(O)—O—(C₁-C₄)-alkyl,
—C(O)—OH,
—(C₁-C₆)-alkyl that is unsubstituted or substituted, once, twice or three times, by halogen,
—O—(C₁-C₆)-alkyl, wherein the alkyl is unsubstituted or substituted, once, twice or three times, by halogen,
pyridyl, or
phenyl that is unsubstituted or substituted, once or more than once and independently of each other, by a radical from the series halogen,
—(C₁-C₄)-alkoxy and —(C₁-C₄)-alkyl.

3. The compound according to claim 1, wherein
R¹ is
hydrogen,
R² is
—(C₁-C₂)-alkyl that is substituted, once, twice or three times, by
phenyl that is substituted, once, twice or three times, independently of each other, by R¹¹, or
Het that is furan, imidazole, isothiazole, isoxazole, oxazole, pyrazole, pyridazine, pyridine, pyrimidine, pyrrole, thiazole, thiophene, 1,2,3-triazole or 1,2,4-triazole, and Het is unsubstituted or substituted, once, twice or three times, independently of each other, by R¹³,
R³, R⁴, R⁵, R⁶ and R⁷ are identical or different and are, independently of each other,
hydrogen,
halogen,
methyl,
trifluoromethyl,
methoxy, or
trifluoromethoxy,
R⁸ is
hydrogen atom, or
—(C₁-C₄)-alkyl,
R¹¹ is
—(C₂-C₄)-alkyl-C(O)—O—R⁸,
—O—(C₁-C₄)-alkyl-C(O)—O—R⁸,
—N R¹⁴R¹⁵, wherein R¹⁴ and R¹⁵ taken together with the nitrogen atom to which they are attached form imidazolidine, isothiazolidine, isoxazolidine, morpholine, piperazine, piperidine, pyrazine, pyrazolidine, pyrrolidine, tetrazine or thiomorpholine, and wherein each nitrogen atom thereof is optionally independently unsubstituted or substituted by (C₁-C₄)-alkyl,
—(CH₂)ₖ—NR⁹R¹⁰,
—O—(C₂-C₄)-alkyl-NR⁹R¹⁰, or
—NH—C(O)—(C₁-C₄)-alkyl, wherein the alkyl is unsubstituted or substituted, once, twice or three times, by R¹²,
R⁹ and R¹⁰ are identical or different and are, independently of each other,
hydrogen atom, or
—(C₁-C₄)-alkyl, or
taken together with the nitrogen atom to which they are attached form imidazolidine, isothiazolidine, isoxazolidine, morpholine, piperazine, piperidine, pyrazine, pyrazolidine, pyrrolidine, tetrazine or thiomorpholine, and wherein the nitrogen atom is optionally unsubstituted or substituted by —(C₁-C₄)-alkyl,
k is
2, 3 or 4,
R¹² is
halogen,
—C(O)—O—(C₁-C₄)-alkyl, or
—C(O)—OH, and
R¹³ is
halogen,
amino,
—C(O)—O—(C₁-C₄)-alkyl,
—C(O)—OH,
—(C₁-C₄)-alkyl that is unsubstituted or substituted, once, twice or three times, by halogen,
—O—(C₁-C₄)-alkyl, wherein the alkyl is unsubstituted or substituted, once, twice or three times, by halogen,
pyridyl, or
phenyl that is unsubstituted or substituted, once or more than once and independently of each other, by a radical from the series halogen,
—(C₁-C₄)-alkoxy and —(C₁-C₄)-alkyl.

4. A method for the prophylaxis or therapy of a patient having or subject to a disease whose course involves a detrimental increase in the activity of matrix metalloproteinase 13, comprising administering to said patient a therapeutically effective amount of a compound according to claim 1.

5. A method for the prophylaxis or therapy of a patient having or subject to a disease whose course involves a detrimental increase in the activity of matrix metalloproteinase 13, comprising administering to said patient a therapeutically effective amount of a compound according to claim 2.

6. A method for the prophylaxis or therapy of a patient having or subject to a disease whose course involves a detrimental increase in the activity of matrix metalloproteinase 13, comprising administering to said patient a therapeutically effective amount of a compound according to claim 3.

7. A process for preparing the compound of formula I according to claim 1, comprising
a) reacting a compound of formula II 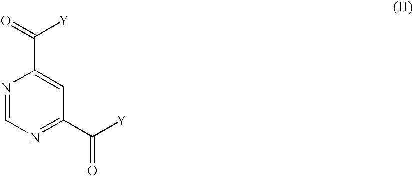
wherein Y is
halogen, hydroxyl or C₁-C₄-alkoxy, or forms, together with the carbonyl group, an active ester or a mixed anhydride,
with a compound of formula IIIa 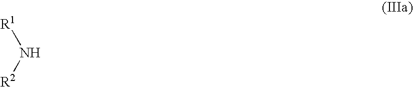
wherein R¹ and R², have the meanings given in the compound of formula I, to form a compound of formula IVa 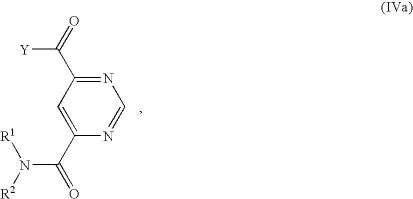
b) reacting the compound of formula IVa with a compound of formula IIIb 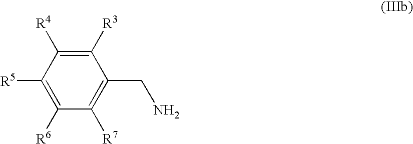
wherein R³, R⁴, R⁵, R⁶ and R⁷ have the meanings given in the compound of formula I, to form the compound of formula I.

8. A process for preparing the compound of formula I according to claim 1, comprising
a) reacting a compound of formula II 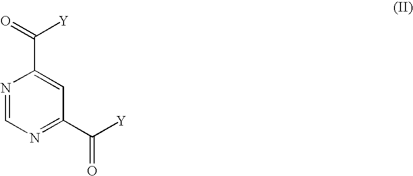
wherein Y is
halogen, hydroxyl or Cl-C₄-alkoxy, or forms, together with the carbonyl group, an active ester or a mixed anhydride,
with a compound of formula IIIb 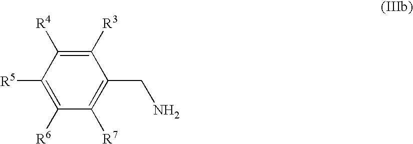
wherein R³, R⁴, R⁵, R⁶ and R⁷ have the meanings given in the compound of formula I,
to form a compound of formula IVb 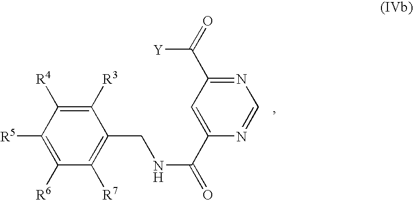
b) reacting the compound of formula IVb with a compound of formula IIIa 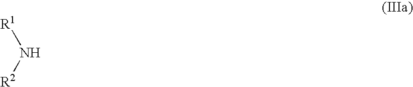
wherein R¹ and R², have the meanings given in the compound of formula I, to form the compound of formula I.

9. A pharmaceutical preparation comprising a pharmaceutically effective amount of at least one compound of formula I according to claim 1 and a pharmaceutically suitable and physiologically tolerated carrier.

10. A use of the compound according to claim 1 for the prophylaxis or therapy of a patient having or subject to a disease that involves a detrimental increase in the activity of matrix metalloproteinase 13, comprising administering to the patient a pharmaceutically effective amount of at least one compound of formula I.

11. The use according to claim 10 wherein the disease is a degenerative joint disease, or disease of the connective tissue, chronic disease of the locomotory apparatus or cancer disease such as breast cancer.
